(19) United States
(12) Patent Application Publication
(10) Pub. No.: US 2025/0127922 A1
(43) Pub. Date: Apr. 24, 2025

(54) CAPSID

(71) Applicant: UCL Business PLC, London (GB)

(72) Inventors: Amit Nathwani, London (GB); Allison Dane, London (GB)

(21) Appl. No.: 18/911,006

(22) Filed: Oct. 9, 2024

Related U.S. Application Data
(62) Division of application No. 16/541,516, filed on Aug. 15, 2019, now Pat. No. 12,138,318.
Application No. 16/541,516 is a continuation of application No. 15/573,350, filed on Nov. 10, 2017, now Pat. No. 10,414,803.

(30) Foreign Application Priority Data
May 11, 2015 (GB) 1508026.0

(51) Int. Cl. A61K 48/00; C07K 14/005; C12N 15/86

(52) U.S. Cl. CPC A61K 48/0008; C07K 14/005; C12N 15/86; C12N 2750/14122; C12N 2750/14143

(57) ABSTRACT

There is described an AAV capsid protein having an amino acid sequence which has at least 98% identity to the sequence of SEQ ID NO: 3 or at least 94% identity to the sequence of SEQ ID NO: 4. Also described is a pharmaceutical composition, an AAV capsid and a viral particle comprising the capsid protein, a recombinant AAV vector comprising a nucleotide sequence which encodes for the capsid protein, and a host cell and a transgenic animal comprising the capsid protein or the vector. In addition, there is described a method of transferring a nucleic acid of interest into a mammal comprising introducing a recombinant AAV vector into the mammal, wherein the recombinant AAV vector comprises a gene of interest which is encapsidated into a capsid comprising the capsid protein.

### CROSS-REFERENCE TO RELATED APPLICATIONS

This application is a divisional of U.S. application Ser. No. 16/541,516, filed on Aug. 15, 2019, which is a continuation of U.S. application Ser. No. 15/573,350, filed Nov. 10, 2017, now U.S. Pat. No. 10,414,803, which is a national phase entry under 35 U.S.C. § 371 of International Patent Application PCT/GB2016/051329, filed May 10, 2016, designating the United States of America and published in English as International Patent Publication WO 2016/181123 A1 on Nov. 17, 2016, which claims the benefit under Article 8 of the Patent Cooperation Treaty to Great Britain Patent Application Serial No. 1508026.0 filed May 11, 2015.

### REFERENCE TO A SEQUENCE LISTING XML

This application contains a Sequence Listing which has been submitted electronically in XML format. The Sequence Listing XML is incorporated herein by reference. Said XML file, created on Dec. 10, 2024, is named 2967278-000007-US3_SL.xml and is 11,839 bytes in size.

### FIELD OF THE INVENTION

The invention relates to adeno-associated virus (AAV) capsid variants.

### BACKGROUND TO THE INVENTION

Multiple recombinant gene transfer vectors based on different viruses have been developed in recent years. Gene transfer vectors based on adeno-associated virus (AAV), i.e., AAV vectors, are preferred due to their ability to transduce different types of dividing and non-dividing cells of different tissues and the ability to establish stable, long-term transgene expression. While vectors based on other viruses, such as adenoviruses and retroviruses may possess certain desirable characteristics, they have also been associated with undesirable side effects. Such side effects have not been detected with gene transfer vectors based on AAV (Manno et al., Nature Medicine, 12 (3): 342 (2006)).

Many AAV serotypes have been identified, cloned, sequenced, and converted into vectors. These serotypes include AAV8, AAV5, AAV3B and the more recently described AAV-LK03 (WO 2013/029030). However, the present inventors have found that many of the currently used vectors have a low transduction rate in humans. For example, AAV8 vectors have a 20-fold lower transduction in humans than in mice as well as transient, prednisolone responsive, transaminitis which occurred in two-thirds of a high dose cohort. Thus, a need remains for new AAV vectors to improve potency and safety, as well as promoting wider clinical applicability.

To this end, the inventors have developed new hybrid capsids by empirically swapping various domains from 4 different AAV capsids: (1) AAV8, (2) AAV5, (3) AAV3B, and (4) AAV-LK03. Capsids were developed which achieved up to a 5-fold higher level of gene transfer than currently used vectors.

### SUMMARY OF THE INVENTION

In a first aspect of the invention, there is provided an AAV capsid protein having an amino acid sequence which has at least 98% identity to SEQ ID NO: 3 or at least 94% identity to SEQ ID NO: 4.

The inventors have surprisingly found that the novel capsid protein produces a capsid which provides a higher transduction rate than currently used AAV vectors. Further, it has been found that the prevalence of antibodies to the capsid in patients is lower than for some currently used AAV vectors.

In some embodiments, the amino acid sequence has at least 95% identity to the sequence of SEQ ID NO: 4. In particular embodiments, the amino acid sequence has at least 96% identity to the sequence of SEQ ID NO: 4. In further embodiments, the amino acid sequence has at least 97% identity to the sequence of SEQ ID NO: 4. In some embodiments, the amino acid sequence has at least 98% identity to the sequence of SEQ ID NO: 4. In other embodiments, the amino acid sequence has at least 99% identity to the sequence of SEQ ID NO: 4. In particular embodiments, the amino acid sequence has the sequence of SEQ ID NO: 4.

In some embodiments, the amino acid sequence has at least 98.5% identity to the sequence of SEQ ID NO: 3, preferably at least 99% identity to the sequence of SEQ ID NO: 3 and more preferably at least 99.5% identity to the sequence of SEQ ID NO: 3. In particular embodiments, the amino acid sequence has the sequence of SEQ ID NO: 3.

In some embodiments, the amino acid sequence has identity to the sequence of SEQ ID NO: 3. In other embodiments, the amino acid sequence has identity to the sequence of SEQ ID NO: 4. In preferred embodiments, the amino acid sequence has the sequence of SEQ ID NO: 4. More preferably, the amino acid sequence has the sequence of SEQ ID NO: 3.

The capsid protein having the amino acid sequence defined above is a functional capsid protein which can form a functional capsid, along with other necessary capsid proteins. A functional capsid is one which can enclose genetic material, enter a cell and transduce the cell with the genetic material. It would be well within the capabilities of a skilled person to determine whether a capsid is functional. For example, the experiments described below in the detailed description of the invention can be used to determine whether a capsid can successfully transduce a cell.

SEQ ID NO: 3 is generated by cloning a 146 amino acid region encompassing the AAV8 VP1 region upstream of the VP2 and VP3 domains of the AAV3B capsid. SEQ ID NO: 4 is generated by cloning the AAV5 VP1 region upstream of the AAV3B VP2 and VP3 regions. Further details of AAV serotypes and capsid proteins is provided below.

A second aspect of the invention provides an AAV capsid comprising the AAV capsid protein described above. An AAV capsid is a protein shell made up of VP1, VP2 and VP3 proteins and which encapsidates (or encloses) the genetic material of the virus.

Further, there is provided a viral particle comprising the AAV capsid protein described above. The viral particle comprises the AAV capsid and the genetic material of the virus.

In a third aspect of the invention, there is provided a recombinant AAV (rAAV) vector comprising a nucleotide sequence which encodes for the amino acid sequence described above. This means that the vector contains a nucleotide sequence encoding for a functional capsid protein.

Preferably the vector further comprises a promoter such that the nucleotide sequence is expressible. Preferably the vector comprises the AAV2 p40 viral promoter, which is constitutionally active in most mammalian cell types.

Accordingly, the present invention provides gene delivery vectors based on adeno-associated virus (AAV), which normally infects humans (e.g., serotypes 1, 2, 3A, 3B, 4, 5, and 6) or primates (e.g., serotypes 1 and 4). Further information on this virus is described in Kenneth I. Berns, “Parvoviridae: The Viruses and Their Replication,” Chapter 69 in Fields Virology (3d Ed. 1996).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, VP2 and VP3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

In an AAV suitable for use as a gene therapy vector, the vector genome typically comprises a nucleic acid of interest to be packaged for delivery to a target cell. According to this particular embodiment, the nucleic acid is located between the viral ITRs at either end of the vector genome. It is possible for an AAV genome to function with only one ITR. Thus, in a gene therapy vector of the invention based on AAV, the vector genome is flanked by at least one ITR, but, more typically, by two AAV ITRs (generally with one either side of the vector genome, i.e. one at the 5′ end and one at the 3′ end). There may be intervening sequences between the nucleic acid in the vector genome and one or more of the ITRs.

In the context of the invention “at least one ITR” is understood to mean a palindromic sequence, comprising mostly complementary, symmetrically arranged sequences also referred to as “A,” “B,” and “C” regions. The ITR functions as an origin of replication, a site having a “cis” role in replication, i.e., being a recognition site for trans-acting replication proteins such as e.g. Rep 78 (or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. An AAV replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. For safety reasons it may be desirable to construct an AAV vector that is unable to further propagate after initial introduction into a cell. Such a safety mechanism for limiting undesirable vector propagation in a recipient may be provided by using AAV with a chimeric ITR as described in US2003148506.

Those skilled in the art will appreciate that the viral Rep protein(s) used for producing an AAV vector of the invention may be selected with consideration for the source of the viral ITRs. For example, the AAV5 ITR typically interacts more efficiently with the AAV5 Rep protein, although it is not necessary that the serotype of ITR and Rep protein(s) are matched.

The ITR(s) used in the invention are typically functional, i.e. they may be fully resolvable and are AAV sequences, with serotypes 1, 2, 3, 4, 5 or 6 being preferred. Resolvable AAV ITRs according to the present invention need not have a wild-type ITR sequence (e.g., a wild-type sequence may be altered by insertion, deletion, truncation or missense mutations), as long as the ITR mediates the desired functions, e.g., virus packaging, integration, and/or provirus rescue, and the like.

In further preferred embodiments, the AAV cap genes and AAV rep genes are deleted from the template genome (and thus from the virion DNA produced therefrom). This configuration maximizes the size of the nucleic acid sequence(s) that can be carried by the AAV capsid.

AAV sequences that may be used in the present invention for the production of AAV gene therapy vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716. AAV serotype 1, 2, 3, 4, 5, 6, 7, 8 or 9 may be used in the present invention. However, AAV serotypes 1, 5 or 8 are preferred sources of AAV sequences for use in the context of the present invention. The sequences from the AAV serotypes may be mutated or engineered when being used in the production of gene therapy vectors.

Preferably, the AAV ITR sequences for use in the context of the present invention are derived from AAV1, AAV2, AAV4 and/or AAV6. Likewise, the Rep (Rep78 and Rep52) coding sequences are preferably derived from AAV1, AAV2, AAV4 and/or AAV6.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82%. Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1, 5 or 8) and the Rep and/or ITR sequences of another AAV serotype (e.g., AAV2). Such pseudotyped rAAV particles are part of the present invention as the Rep and/or ITR sequences of any AAV serotype can be used with the modified capsid protein of the invention.

Modified “AAV” sequences also can be used in the context of the present invention, e.g. for the production of AAV gene therapy vectors. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, or Rep can be used in place of wild-type AAV ITR, or Rep sequences.

Although similar to other AAV serotypes in many respects, AAV5 differs from other human and simian AAV serotypes more than other known human and simian serotypes. In view thereof, the production of rAAV5 can differ from production of other serotypes in insect cells. Where methods of the invention are employed to produce rAAV5, it is preferred that one or more constructs comprising, collectively in the case of more than one construct, a nucleotide sequence comprising an AAV5 ITR, a nucleotide sequence comprises an AAV5 Rep coding sequence (i.e. a nucleotide sequence comprises an AAV5 Rep78). Such ITR and Rep sequences can be modified as desired to obtain efficient production of AAV5 or pseudotyped AAV5 vectors.

It is within the technical skills of the skilled person to select the most appropriate virus, virus subtype or virus serotype. Some subtypes or serotypes may be more appropriate than others for a certain type of tissue.

For example, liver-specific expression of a nucleic acid of interest may advantageously be induced by AAV-mediated transduction of liver cells. Liver is amenable to AAV-mediated transduction, and different serotypes may be used (for example, AAV1, AAV5 or AAV8). Transduction of muscle may be accomplished by administration of an AAV encoding a nucleic acid via the blood stream. Thus, intravenous or intra-arterial administration is applicable.

Advantageously, by using the vector of the present invention, a greater degree of cellular transduction can be obtained. This is particularly the case in liver cells.

Accordingly, the vectors of the invention therefore represent a tool for the development of strategies for the in vivo delivery of a therapeutic nucleotide sequence, by engineering the nucleic acid within a gene therapy vector that efficiently transduces an appropriate cell type, such as a liver cell.

The vector may comprise other elements to allow the functional therapeutic protein to be expressed. Such elements are well known to a person skilled in the art.

Preferably, the nucleic acids and amino acid sequences described above are isolated.

It would be well with the capabilities of a skilled person to produce the nucleic acid molecules and amino acid sequences described above. This could be done, for example, using chemical synthesis of a given sequence.

The invention also relates to a method of transferring a nucleic acid of interest into a mammal comprising introducing a recombinant AAV vector into a mammal, the recombinant AAV vector comprising a gene of interest which is encapsidated into a capsid comprising the capsid protein described above.

The invention also provides a host cell comprising the capsid protein having the amino acid sequences described above or the vectors described above.

As used herein, the term “host” refers to organisms and/or cells which harbour a protein or a vector of the invention, as well as organisms and/or cells that are suitable for use in expressing a recombinant gene or protein. It is not intended that the present invention be limited to any particular type of cell or organism. Indeed, it is contemplated that any suitable organism and/or cell will find use in the present invention as a host. A host cell may be in the form of a single cell, a population of similar or different cells, for example in the form of a culture (such as a liquid culture or a culture on a solid substrate), an organism or part thereof.

A host cell according to the invention may permit the expression of a therapeutic nucleic acid molecule. Thus, the host cell may be, for example, a bacterial, a yeast, an insect or a mammalian cell.

In addition, the invention provides a transgenic animal comprising cells comprising the capsid protein having the amino acid sequence described above or a vector described above. Preferably the animal is a non-human mammal, especially a primate. Alternatively, the animal may be a rodent, especially a mouse; or may be canine, feline, ovine or porcine.

In one aspect, the invention provides a pharmaceutical composition comprising the capsid protein having the amino acid sequence of the present invention or a vector of the invention and one or more pharmaceutically acceptable excipients. The one or more excipients include carriers, diluents and/or other medicinal agents, pharmaceutical agents or adjuvants, etc.

The higher levels of gene transfer achieved with the sequences of the present invention represents an advance beyond the current state of the art. An improvement in potency has been observed which will permit gene transfer using lower vector doses, thus improving safety (especially reducing liver toxicity which is dose dependent) and at a lower cost. Accordingly the sequences and vector of the present invention may have a clinical applicability to disorders such as congenital FVII deficiency, Gaucher's disease, OTC deficiency, Fabry's disease, glycogen storage diseases, α-1-antitrypsin deficiency, progressive familial intrahepatic cholestasis, Wilson's disease, Crigler Najjar syndrome and hepatocellular carcinoma amongst others.

Accordingly, the invention may provide a method of treating one or more of the diseases listed above comprising administering a therapeutically effective amount of a vector as described above to a patient suffering from said disease. Preferably, the patient is human.

When said disease is “treated” in the above method, this means that one or more symptoms of said disease are ameliorated. It does not mean that the symptoms of said disease are completely remedied so that they are no longer present in the patient, although in some methods, this may be the case. The method of treating results in one or more of the symptoms of said disease being less severe than before treatment.

A “therapeutically effective amount” refers to an amount effective, at dosages and for periods of time necessary, to achieve the desired therapeutic result.

Further, the invention may provide the capsid protein as described above, or a vector as described above for use in therapy, preferably for one of the afore-mentioned diseases.

In addition, the invention may provide for the use of the capsid protein as described above or a vector as described above in the manufacture of a medicament for treating one of the diseases as mentioned above.

In the description above, the term “identity” is used to refer to the similarity of two sequences. For the purpose of this invention, it is defined here that in order to determine the percent identity of two amino acid sequences, the sequences are aligned for optimal comparison purposes (e.g., gaps can be introduced in the sequence of a amino acid for optimal alignment with a second amino acid sequence). The amino acid residues are then compared. When a position in the first sequence is occupied by the same amino acid or amino acid residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

A sequence comparison may be carried out over the entire lengths of the two sequences being compared or over fragment of the two sequences. Typically, the comparison will be carried out over the full length of the two sequences being compared. However, sequence identity may be carried out over a region of, for example, about twenty, about fifty, about one hundred, about two hundred, about five hundred, about 1000 or about 2000 or more contiguous amino acid residues.

The skilled person will be aware of the fact that several different computer programs are available to determine the homology or identity between two sequences. In preferred embodiments, the identity between two sequences is analysed using the software package Clone Manager Professional version 9 (preferably, version 9.4). This analysis tool is produced by Sci-Ed Software (Scientific & Educational Software, 11010 Lake Grove Blvd, Ste 100, PMB 122, Morrisville, NC 27560, USA—http://www.scied.com/index.htm). The settings used to compare the sequences are preferably as follows: alignment: Global DNA alignment; parameters: both strands; scoring matrix: linear (mismatch 2, OpenGap 4, ExtGap 1). Alternatively methods such as Fast Scan—MaxScore and Fast Scan MaxQual can also be used with the same software and local settings.

Other methods can also be used to determine sequence identity. For example, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

All patent and literature references cited in the present specification are hereby incorporated by reference in their entirety.

A skilled person will appreciate that all aspects of the invention, whether they relate to, for example, the amino acid, the viral particle, vector, the host cell or the use, are equally applicable to all other aspects of the invention. In particular, aspects of the method of treatment, for example, the administration of the vector, may have been described in greater detail than in some of the other aspects of the invention, for example, relating to the use of the vector in therapy. However, the skilled person will appreciate where more detailed information has been given for a particular aspect of the invention, this information is generally equally applicable to other aspects of the invention. Further, the skilled person will also appreciate that the description relating to the method of treatment is equally applicable to the use of vector in therapy.

### DETAILED DESCRIPTION OF THE INVENTION

The invention will now be described in detail by way of example only with reference to the figures in which:

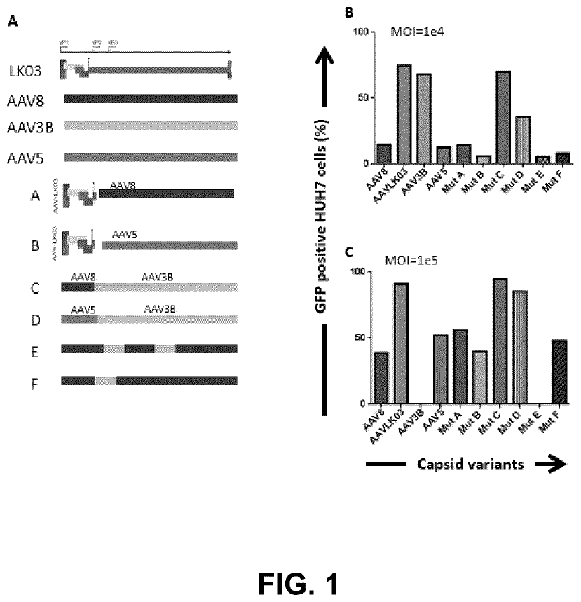

FIG. 1 shows the evaluation of hybrid AAV capsids. Part A shows a schematic of the hybrid capsids and part B illustrates the in vitro transfer efficiency of the different hybrid capsids at two different MOIs (Multiplicity of Infections) in HUH7 cells.

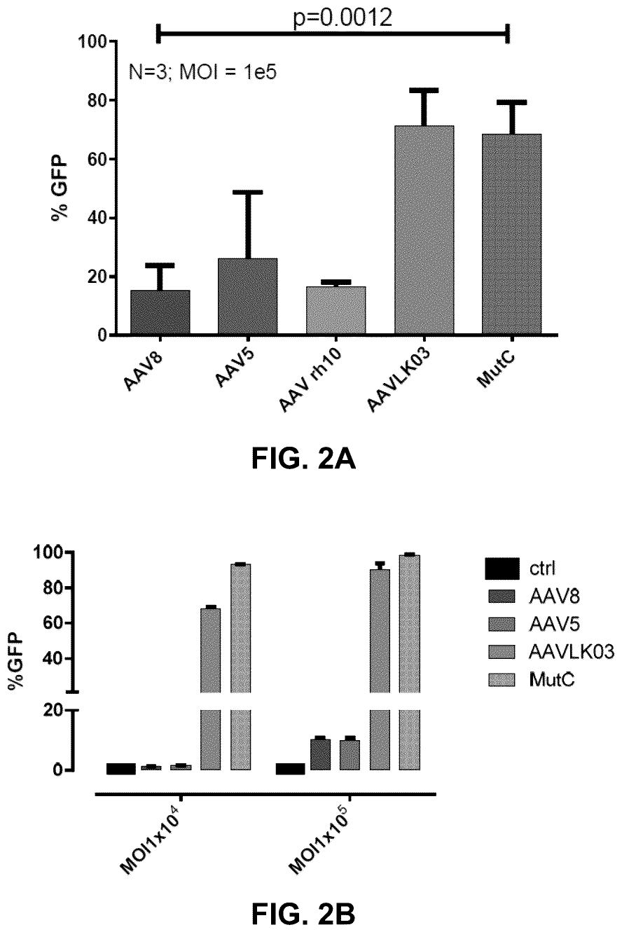

FIG. 2A and FIG. 2B show the results of cell transduction experiments.

FIG. 2A shows the results of transduction of HUH7 cells with AAV8, AAV5, AAV-rh10, AAV-LK03 and AAV Mutant C vectors. FIG. 2B shows the results of transduction of adherent HepG2 cells with AAV8, AAV5, AAV-LK03 and AAV Mutant C vectors.

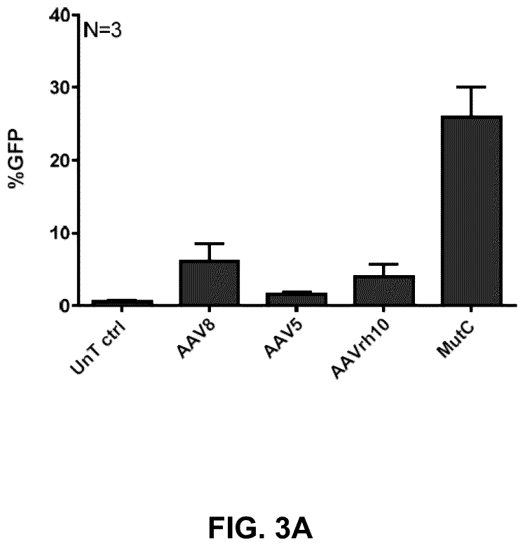

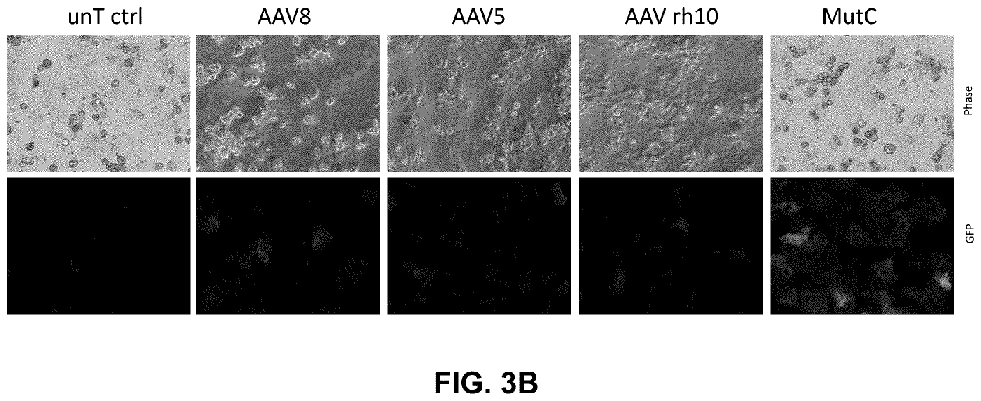

FIG. 3A shows the flow-cytometric analysis of the transduction of primary hepatocytes with AAV8, AAV5, AAV-rh10 and AAV Mutant C vectors. FIG. 3B is fluorescent microscope images of the transduced hepatocytes.

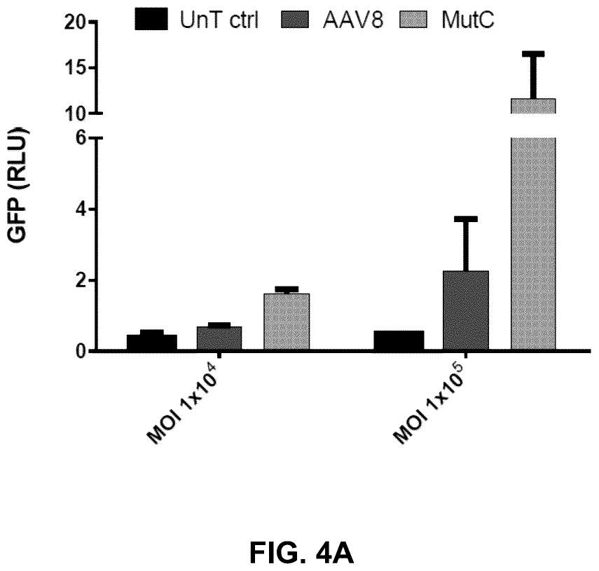

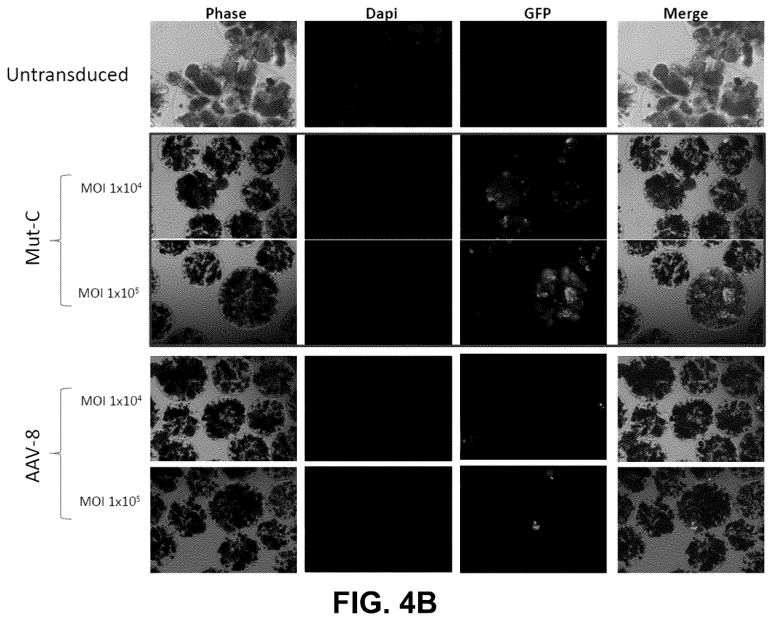

FIG. 4A shows the GFP excitation/emission at 485 nm/535 nm resulting from transduction of 3D cultures of HepG2 cells with AAV8 and AAV Mutant C vectors containing a GFP gene at a MOI of 1×10⁴ or 1×10⁵. FIG. 4B is fluorescent microscope images of the transduced 3D HepG2 cells (5× objective, GFP exposure time constant [300 ms]).

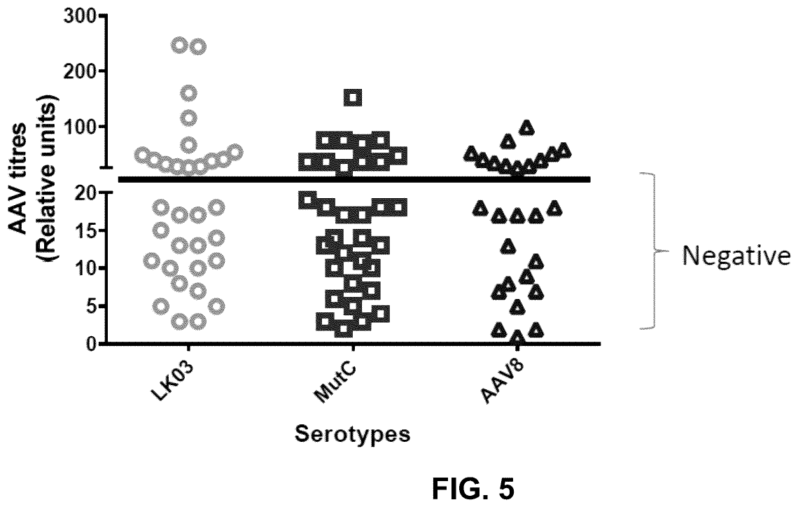

FIG. 5 shows the prevalence of antibodies to AAV vectors in severe haemophilia patients as a result of an ELISA immune-adsorption method.

The inventors' research program was designed to establish an AAV vector which has a relatively high transduction rate and minimal side effects. As a result, the inventors have developed new hybrid capsids and vectors.

### Example 1

New hybrid capsids were generated by empirically swapping various domains from 4 different AAV capsids: (1) AAV8, (2) AAV5, (3) AAV3B, and (4) AAV-LK03 (see FIG. 1). The latter has been reported to transduce human hepatocytes more efficiently than the currently available naturally occurring AAV capsids. Notably, LK03 has >95% amino acid sequence homology with AAV3B. The capsid protein of the present invention is a synthetic capsid that has been developed by selection of motifs from wild type AAV capsids from serotypes 1-rh10. The VP2 and VP3 regions of LK03 were replaced with corresponding regions from AAV8 and AAV5 to generate mutants A (SEQ ID NO: 1) (Mut A) and B (SEQ ID NO: 2) (Mut B) respectively. In mutant C (SEQ ID NO: 3) (Mut C), a 146 amino acid region encompassing the AAV8 VP1 region was cloned upstream of the VP2 and VP3 domains of the AAV3B capsid. Similarly, in mutant D (SEQ ID NO: 4) (Mut D) the AAV5-VP1 region was cloned upstream of the AAV3B VP2 and VP3 regions. In mutant E (SEQ ID NO: 5) (Mut E), domains v-I, v-II, and v-IX of AAV8 were replaced with cognate from AAV3B. In mutant F (SEQ ID NO: 6) (Mut F), a 262 amino acid region containing the hepatocyte growth factor receptor binding site of AAV3B was cloned into the corresponding region of the AAV8 capsid.

The ratio of VP1, VP2 and VP3 for the synthetic capsids, in particular mutant C and mutant D, is similar to that observed for AAV2, 3b, 5 or 8. Both mutant C and mutant D have a strong tropism for human liver cells and are able to mediate higher levels of gene transfer into these cells when compared to wild type AAV capsids including AAV5 and AAV8.

The capsid sequences were assembled by overlapping PCR or generated by gene synthesis (GenScript; Mut C, Mut E and F). The capsid sequences were cloned into an AAV helper plasmid containing the Rep78 coding sequence of AAV2 which has a modified start codon (ATG to ACG). Vector stocks were prepared by standard triple plasmid transfection of HEK293 cells with pCMV-eGFP, pRep2Cap and the adenoviral helper plasmid, pHGTI. Vector was purified by density gradient centrifugation with iodixanol. Vector genomes were titered by QPCR with primers specific for eGFP. The yield of the hybrid AAV vectors was comparable to that previously observed with AAV8. Gene transfer efficiency of these hybrid capsids was assessed in vitro using the HUH7 hepatocellular carcinoma cell line at multiplicity of infection (MOI) 1×10⁴ and 1×10⁵. Level of gene transfer was determined by detection of the reporter gene, eGFP, by flow cytometry. The highest level of gene transfer was achieved with mutant C at levels that were at least 5-fold higher than AAV8 (FIG. 1B). This mutant mediated similar levels of gene transfer as AAV3B and novel serotype LK03. Efficiency of gene transfer of mutant D was lower than that of mutant C but 3-fold higher than parental serotype, AAV5. Mutants E and F had much lower levels of gene transfer when compared to mutant C with similar levels of GFP positive cells as seen with wild type AAV8 and AAV5.

### Example 2

Production of recombinant AAV vectors. AAV vectors were made by co-transfection of adherent HEK293 T-cells with a combination of plasmids consisting of the vector plasmid in which the EGFP reporter gene was under the control of CMV promoter, an adenoviral helper plasmid, and a packaging plasmids in which the respective AAV cap gene was downstream of AAV2 Rep gene under the control of the endogenous promoters. Vectors were purified using AVB column chromatography. Titration of all vectors was performed by qPCR assay as well as alkaline gel analysis.

In-vitro transduction. Liver cancer cell lines grown in monolayer were transduced with AAV at various MOI's followed by assessment of transduction efficiency using flow cytometry at ˜72 hours after gene transfer.

Primary human hepatocytes were obtained from Life-technologies and maintained in culture as per supplier's instruction. They were then exposed to AAV encoding GFP under the CMV promoter. Gene transfer efficiency was assessed 3-4 days later using either flow cytometry or direct fluorescent microscopy.

3D cultures of encapsulated HepG2 cells were exposed to different MOI's of AAV followed by assessment of by quantify green fluorescence using a plate reader or by direct fluorescent microscopy.

Anti-AAV-antibody titre. An immunoabsorption method was used to assess anti-AAV antibody titre in plasma samples obtained from severe haemophilia patients. Anti-AAV antibody titres were expressed as the end-point titre (Relative units/ml) defined as the reciprocal of the interpolated dilution with an absorbance value equal to five times the mean absorbance background value.

### Transduction Efficiency of AAV Mutant C Vectors Compared to Other Serotypes in Human Liver Cells Lines and Primary Human Hepatocytes

In a focused study, the in vitro transduction efficiency of AAV8, AAV5, AAV-rh10, AAV-LK03 and AAV Mut C in Huh7 (liver cancer cell line) and HepG2 cell lines (liver cancer cell line) was compared. All vectors contained the green fluorescent protein (GFP) reporter gene under the control of the cytomegalovirus (CMV) promoter.

AAV8, AAV5 and AAV-rh10 vectors were less efficient at transducing Huh7 cells with mean GFP expression ranging from 15%-26%. In comparison higher levels of gene transfer were observed with AAV-LK03, and Mut C with transduction efficiency approaching 70% for vectors pseudotyped with these capsid proteins (FIG. 2A). The difference in gene transfer efficiency between Mut C and AAV8 was highly significant (p=0.0012).

Similar results were obtained in the HepG2 cell line (FIG. 2B) with almost 10 fold higher gene transfer efficiency observed when HepG2 cells were transduced with AAV Mut C and AAV-LK03 when compared to AAV8 and AAV5, irrespective of MOI (P=<0.05).

Next gene transfer efficiency of primary human hepatocytes derived from 3 different donors was assessed. All donors were male with 2 being of Caucasian origin and 1 of Afro-Caribbean origin. The age of these donors ranged from 21-52 years. Primary hepatocytes were exposed to AAV vector at an MOI of 106 using a vector encoding GFP under the control of CMV promoter. Transduction efficiency was assessed by fluorescent microscopy or flow cytometry at 4-5 days after gene transfer. As shown in FIG. 3, gene transfer efficiency achieved with AAV Mut C (32.06±4%) was more than 4× higher than that achieved with AAV8 (7.3±7%). The difference in gene transfer efficiency of primary human hepatocytes between AAV Mut C and AAV8 groups was highly significant (p=0.0015 using 1 sample t-test). Direct fluorescent image analysis validated the flow cytometric data showing a much higher level of GFP expression in the cells transduced with AAV Mut C (FIG. 3B).

The inventors next looked at AAV mediated gene transfer into 3D cultures of HepG2 cells encapsulated in alginate using a JetCutter to produce approximately 500 μm spherical beads containing HepG2 cells at an approximate concentration of 1.7M per ml beads. The strength of this model is that within the beads encapsulated cells proliferate to form compact cell spheroids (AELS) with good cell to cell contact and cell function. AELS exhibit better growth and upregulated liver specific function compared to monolayer cultures, with typical cell architecture demonstrating desmosomes, tight junctions and micro-villi, high representation of endoplasmic reticulum and mitochondria, and generation of extracellular matrix reminiscent of that in normal liver.

The 3D bead cultures were transduced with either AAV8 or AAV Mut C at an MOI of 1×10⁴ or 1×10⁵ using a vector containing the CMV GFP expression cassette. As before transduction efficiency was determined by quantify green fluorescence using a plate reader (FIG. 4A) or direct fluorescent microscopy (FIG. 4B). A 3-5 fold higher gene transfer efficiency of the 3D spheroids was observed with AAV Mut C when compared to GFP expression in beads transduced with AAV8. This difference in gene transfer efficiency was also noted using direct florescent microscopy.

### Assessment of Pre-Existing Antibodies Against Mutant C in Severe Haemophilia Patients

Plasma samples from unselected severe haemophilia patients were screened to assess the prevalence of pre-existing antibodies against AAV Mut C using an ELISA immune-adsorption method. 63% of the patients displayed no detectable antibodies against AAV Mut C. The corresponding levels for AAV8 and AAV-LK03 were 51%. This therefore suggests that fewer patients with severe haemophilia have antibodies to AAV Mut C.

What is claimed is:

1. An AAV capsid protein having an amino acid sequence which has at least 98% identity to the sequence of SEQ ID NO: 3 or at least 94% identity to the sequence of SEQ ID NO: 4.

2. The AAV capsid protein of claim 1, wherein the amino acid sequence has at least 99% identity to the sequence of SEQ ID NO: 3 or at least 97% identity to the sequence of SEQ ID NO: 4.

3. The AAV capsid protein of claim 1 or claim 2, wherein the amino acid sequence has the sequence of SEQ ID NO. 3.

4. The AAV capsid protein of claim 1 or claim 2, wherein the amino acid sequence has the sequence of SEQ ID NO. 4.

5. An AAV capsid comprising the capsid protein of any one of claims 1 to 4.

6. A viral particle comprising the capsid protein of any one of claims 1 to 4.

7. A recombinant AAV vector, comprising a nucleotide sequence which encodes for the capsid protein of any one of claims 1 to 4.

8. A method of transferring a nucleic acid of interest into a mammal comprising introducing a recombinant AAV vector into a mammal, the recombinant AAV vector comprising a gene of interest which is encapsidated into a capsid comprising the capsid protein of any one of claims 1 to 4.

9. A host cell comprising the capsid protein of any one of claims 1 to 4 or the vector of claim 7.

10. A transgenic animal comprising cells comprising the capsid protein of any one of claims 1 to 4 or the vector of claim 7.

11. A pharmaceutical composition comprising the capsid protein of any one of claims 1 to 4 or the vector of claim 7, and one or more pharmaceutically acceptable excipients.


SEQUENCE LISTING (6 sequences)

SEQ ID NO: 1 - protein, 738 aa, synthetic construct
  source 1..738: note = AAV-LK03, AAV5 and AAV8 derived capsid protein (Mut A)
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 725 aa, synthetic construct
  source 1..725: note = AAV-LK03, AAV5 and AAV8 derived capsid protein (Mut B)
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - protein, 736 aa, synthetic construct
  source 1..736: note = AAV3B and AAV8 derived capsid protein (Mut C)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 735 aa, synthetic construct
  source 1..735: note = AAV3B and AAV5 derived capsid protein (Mut D)
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 5 - protein, 736 aa, synthetic construct
  source 1..736: note = AAV3B and AAV8 derived capsid protein (Mut E)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQGTTASNAELDNVMITDEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYNKSVNVDFTVDTNGVYSEPRPIGTRYLTRNL

SEQ ID NO: 6 - protein, 736 aa, synthetic construct
  source 1..736: note = AAV3B and AAV8 derived capsid protein (Mut F)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL